(19) United States
(12) Patent Application Publication
(10) Pub. No.: US 2026/0318884 A1
(43) Pub. Date: Oct. 15, 2026

(54) DIRECT AFFINITY TO ION EXCHANGE CHROMATOGRAPHY METHODS AND SYSTEMS THEREOF

(71) Applicant: Waters Technologies Corporation, Milford, MA (US)

(72) Inventors: Beatrice Muriithi, Attleboro, MA (US); Kevin Wyndham, Worcester, MA (US); Martin Gilar, Franklin, MA (US)

(73) Assignee: Waters Technologies Corporation, Milford, MA (US)

(21) Appl. No.: 19/644,092

(22) Filed: Apr. 10, 2026

Related U.S. Application Data
(60) Provisional application No. 63/786,450, filed on Apr. 10, 2025.

(51) Int. Cl. G01N 30/14; G01N 30/60

(52) U.S. Cl. CPC G01N 30/14; G01N 30/6039

(57) ABSTRACT

The present disclosure is directed to methods of performing direct affinity-ion exchange (IEX) chromatography, wherein there is a direct elution of the sample from the affinity chromatography column to the IEX column. The methods described herein allow for rapid and robust purification of target analytes from heterogeneous samples, and mitigate the need for complicated valve switching, buffer exchange, or other sample manipulation.

### CROSS-REFERENCE TO RELATED APPLICATIONS

This application claims priority from and the benefit of U.S. Provisional Application No. 63/786,450 filed Apr. 10, 2025. The entire contents of this application are incorporated herein by reference.

### FIELD OF INVENTION

The present disclosure relates generally to methods of performing direct affinity chromatography to ion exchange chromatography, particularly using a direct fluidic connection between the affinity chromatography column and the ion exchange column.

### BACKGROUND

Chromatography methods are used across the pharmaceutical, biotechnology, and chemical industries for the separation and characterization of target analytes in heterogeneous samples. Multiple chromatography methods may be necessary in a particular workflow. For example, the use of affinity chromatography to isolate a target analyte based on its affinity to an affinity agent, and ion exchange chromatography to isolate a target analyte based on its affinity to an ion exchange agent. Typically, the use of both affinity chromatography and ion exchange chromatography requires separate or complex chromatography systems due to technical incompatibilities, including buffer and pressure incompatibilities. In particular, the different solvent conditions required for affinity chromatography and ion exchange chromatography greatly increase the processing required for sample purification method requiring both affinity and ion exchange chromatography. As such, there exists a need in the art for new methods and systems that provide compatibility between affinity and ion exchange chromatography.

### SUMMARY OF INVENTION

Disclosed herein are methods of performing direct affinity-ion exchange chromatography (affinity-IEX chromatography). The methods allow for the direct elution of a sample from an affinity chromatography column onto an IEX column, without the need for valve switching, buffer exchange, or other sample manipulation. That is, the present technology provides affinity chromatography and IEX columns that are connected in direct fluidic connection, preferably via a zero dead volume union. The technology utilizes an affinity chromatography column having a stationary phase including nonporous polymer particles having an average particle size between 1.0 μm to 10 μm and a functionalized surface. Said column affords the elution of a target analyte in a small volume, which can then be directly eluted onto the IEX column without the need for buffer exchange or sample concentration. Further, the affinity chromatography columns and the IEX columns used in the disclosed methods have pressure compatibility, eliminating the need for complicated valve switching designs in the liquid chromatography systems. The present invention also includes methods utilizing a gradient mobile phase (i.e., a mobile phase whose composition is adjusted in-experiment between the affinity chromatography step and the IEX chromatography step).

Accordingly, in a first aspect, the present disclosure provides for a method of purifying a target analyte, the method including:
a) loading a sample including the target analyte onto an affinity chromatography column in direct fluidic connection to an ion exchange chromatography (IEX) column, the affinity chromatography column including:
a plurality of nonporous polymer particles having an average particle size between 1.0 μm to 10 μm; and
one or more affinity agents conjugated to a surface of each particle of the plurality of nonporous polymer particles;
and the IEX column including:
a plurality of IEX particles having an average particle size of between 1 μm and 10 μm and a charged surface, wherein the plurality of particles are nonporous, porous, or superficially porous and the plurality of particles comprise an inorganic material, an organic material, or a hybrid organic-inorganic material;
b) eluting the target analyte from the affinity chromatography column with an affinity buffer directly onto the IEX column; and
c) eluting the target analyte from the IEX column with an IEX buffer;
wherein the IEX buffer includes the affinity buffer and one or more additional components.

In some embodiments, the one or more affinity agents are conjugated directly to a surface of each particle of the plurality of nonporous polymer particles. In some embodiments, the one or more affinity agents are conjugated to the surface of each particle of the plurality of nonporous polymer particles covalently.

In some embodiments, the one or more affinity agents are conjugated indirectly to the surface of each particle through one or more conjugating molecules. In some embodiments, the one or more conjugating molecules include streptavidin. In some embodiments, the affinity agent is biotinylated.

In some embodiments, the one or more affinity agents include an immunoglobulin-binding protein, an antibody or antigen-binding fragment thereof, or an oligonucleotide. In some embodiments, the immunoglobulin-binding protein is Protein A, Protein G, Protein A/G, Protein L, or a binding domain thereof. In some embodiments, the antibody or antigen-binding fragment thereof binds to insulin, AAV9, AAV2, tacrolimus, troponin, IgG, a cytokine, a host cell protein, a dsRNA, or perfluoroalkyl substances (PFAS). In some embodiments, the oligonucleotide is a poly-T oligonucleotide.

In some embodiments, the affinity buffer includes sodium phosphate, sodium chloride, ammonium acetate, or ammonium formate. In some embodiments, the affinity buffer has a pH of from about 6.8 to about 8. In some embodiments, the affinity buffer further includes an organic solvent. In some embodiments, the organic solvent is at a concentration of between 1 and 20% by volume. In some embodiments, the organic solvent is at a concentration of between 1-10% by volume. In some embodiments, the concentration of organic solvent is insufficient to denature the affinity agent. In some embodiments, the organic solvent is ethanol or acetonitrile.

In some embodiments, the IEX buffer includes a salt, wherein the salt is sodium chloride, ammonium acetate, ammonium formate, or ammonium bicarbonate. In some embodiments, the IEX buffer has a pH of from about 5 to about 8. In some embodiments, the method further includes repeating step (b) one or more times, wherein each repetition of step (b) is followed by one or more repetitions of step (c).

In some embodiments, the affinity chromatography column and the IEX column are connected to a high-performance liquid chromatography (HPLC) system, ultra-high performance liquid chromatography (UHPLC) system, or fast protein liquid chromatography (FPLC) system.

In some embodiments, the method further includes the step of detecting the target analyte with a detector after eluting the target analyte from the IEX column. In some embodiments, the detector is an ultraviolet spectroscopy detector, a fluorescence spectroscopy detector, a multi-angle light scattering detector, a charged aerosol detector, and/or a mass spectrometry detector. In some embodiments, the direct fluidic connection is a zero dead volume union.

In a second aspect, the present disclosure further provides for a chromatographic system including:
(a) an affinity chromatography column and an ion exchange chromatography (IEX) column, wherein the affinity chromatography column and the IEX column are connected via a zero dead volume union, wherein the IEX column is positioned downstream of the affinity chromatography column; wherein the affinity chromatography column includes:
a plurality of nonporous polymer particles having an average particle size between 1.0 μm to 10 μm; and
one or more affinity agents conjugated to a surface of the nonporous polymer particle;
and the IEX column includes:
a plurality IEX particles having an average particle size of between 1 μm and 10 μm and a charged surface, wherein the plurality of particles are nonporous, porous, or superficially porous and the plurality of particles comprise an inorganic material, an organic material, or a hybrid organic-inorganic material;
(b) a column injector positioned upstream of the affinity chromatography column; and
(c) tubing in fluidic connection with the IEX column positioned downstream of the IEX column.

In some embodiments, the one or more affinity agents are conjugated directly to a surface of each particle of the plurality of nonporous polymer particles. In some embodiments, the affinity agent is conjugated to the surface of each particle of the plurality of nonporous polymer particles covalently.

In some embodiments, the affinity agent is conjugated indirectly to the surface of each particle through one or more conjugating molecules. In some embodiments, the one or more conjugating molecules include streptavidin. In some embodiments, the affinity agent is biotinylated.

In some embodiments, the one or more affinity agents include an immunoglobulin-binding protein, an antibody or antigen-binding fragment thereof, or an oligonucleotide. In some embodiments, the immunoglobulin-binding protein is Protein A, Protein G, Protein A/G, Protein L, or a binding domain thereof. In some embodiments, the antibody or antigen-binding fragment thereof binds to insulin, AAV9, AAV2, tacrolimus, troponin, IgG, a cytokine, a host cell protein, a dsRNA, or perfluoroalkyl substances (PFAS). In some embodiments, the oligonucleotide is a poly-T oligonucleotide.

In some embodiments, the chromatographic system further includes a detector in fluid communication with the tubing downstream of the IEX chromatography column.

### DEFINITIONS

In order that the methods and technology may be more readily understood, certain terms are first defined. In addition, it should be noted that whenever a value or range of values of a parameter are recited, it is intended that values and ranges intermediate to the recited values are also part of this disclosure. The word “about”, if not defined otherwise, means ±5%. It is also to be noted that as used herein and in the claims, the singular forms “a” and “the” include plural references unless the context clearly dictates otherwise.

As used herein, the term “direct fluidic connection” refers to a flow path between one or more column bodies, column injectors, detectors, and/or tubing connecting said elements, that is uninterrupted by a switching valve.

As used herein, the term “zero dead volume union” refers to a union between one or more elements having a direct fluidic connection, wherein the union introduces 1 μL or less of volume to the flow path. A zero dead volume union can be introduced between any two elements having a direct fluidic connection. In preferred embodiments of the present technology, a zero dead volume union is introduced between an affinity chromatography column and an ion exchange chromatography (IEX) column having a direct fluidic connection.

As used herein, the term “low volume union” refers to a union between one or more elements having a direct fluidic connection, wherein the union introduces 30 uL or less of volume to the flow path, preferably 20 uL or less, more preferably 10 uL or less. A low volume union can be introduced between any two elements having a direct fluidic connection. In some embodiments of the present technology, a low volume union is introduced between an affinity chromatography column and an IEX column having a direct fluidic connection.

As used herein, the term “direct affinity-ion exchange chromatography” or “direct affinity-IEX”, used interchangeably, refers to a method of performing chromatography wherein a sample is first separated via an affinity chromatography column and then directly transferred onto an IEX column, and separated by the IEX column. In the direct affinity-IEX methods described herein, the affinity chromatography column is in direct fluidic connection to the ion exchange chromatography column. That is, any sample injected onto the affinity chromatography column flows through both the affinity chromatography column and the ion exchange chromatography column, as shown in FIG. 1A.

As used herein, the term “eluting the target analyte from the affinity chromatography column” refers to the use of an elution buffer and elution method (such as, e.g., a gradient elution, a single injection elution, or a step elution) to elute analytes bound to the affinity column. In some embodiments, the analytes are eluted in a single narrow peak having a peak volume of between 5-20 μL.

As used herein, the term “conjugate” refers to the linkage of two molecules formed by the chemical bonding of a reactive functional group of one molecule with an appropriately reactive functional group of another molecule. Nonporous polymer particles may have one or more affinity reagents conjugated to the surface of said particles. For example, one or more affinity agents, such as Protein A, may be conjugated directly to a particle via an interaction with an epoxide on the surface of the particle. Alternatively, an affinity agent may be indirectly conjugated to the surface of the nonporous polymer particles via a linker (such as a polyethylene glycol (PEG) linker) or via an interaction with a streptavidin molecule. For the latter instance, one or more streptavidin molecules are conjugated directly to a particle via an interaction with an epoxide on the surface of the particle, which can then bind, via an ionic interaction, to a biotinylated affinity agent.

As used herein, the term “antibody” refers to an immunoglobin molecule that specifically binds to, or is immunologically reactive with, a particular antigen. This includes polyclonal, monoclonal, genetically engineering, and otherwise modified forms of antibodies, including but not limited to chimeric antibodies, camelids, monobodies, humanized antibodies, heteroconjugate antibodies (e.g., bi-, tri-, and quad-specific antibodies, diabodies), and antigen-binding fragments of antibodies, including, for example, Fab′, F(ab′)2, Fab, Fv, and scFv fragments. Unless otherwise indicated, the term “monoclonal antibody” is meant to include both intact molecules as well as antibody fragments that are capable of specifically binding to a target protein. As used herein, the Fab and F(ab′)2 fragments refer to antibody fragments that lack the Fc portion of an intact antibody.

As used herein, the term “polyclonal antibody” refers to an antibody or a population of antibodies that has specificity to one or more antigens (such as, e.g., host cell proteins from a host cell line). A population of polyclonal antibodies recognize one or more distinct epitopes of the one or more antigens.

As used herein, the term “antigen-binding fragment” refers to one or more fragments of an antibody that retain the ability to specifically bind to a target antigen. The antigen-binding function of an antibody can be performed by fragments of a full-length antibody. The antibody fragments can be, for example, a Fab, F(ab′)₂, scFv, a camelid, an affibody, a nanobody, an aptamer, or a domain antibody.

As used herein, the term “bispecific antibody” refers to an antibody that is capable of binding at least two different antigens.

The term “nonporous” or “nonporous core” as used herein, refers to a material or a material region (e.g., the core) that has a pore volume that is less than 0.1 cc/g. Preferably, nonporous polymer cores have a pore volume that is less than 0.10 cc/g (e.g., 0.05 cc/g), and preferably less than 0.02 cc/g, in some embodiments. Pore volume is determined using methods known in the art based on multipoint nitrogen sorption experiments (Micromeritics ASAP 2400; Micromeritics Instruments Inc., Norcross, GA).

### BRIEF DESCRIPTION OF THE DRAWINGS

FIGS. 1A-1C depicts a liquid chromatography system and flow path for performing direct affinity-IEX according to embodiments of the technology.

FIG. 1A shows the flow path across an LC system set up according to embodiments of the technology.

FIG. 1B provides an enlarged view of the zero dead volume or low volume union between an affinity chromatography column and an IEX column.

FIG. 1C provides an exemplary zero dead volume union.

FIG. 2A provides elution results of a NISTmAb in buffer sample from a Protein A affinity column.

FIG. 2B provides the elution results from an ion exchange column of the same NIST mAb in buffer sample as used in the elution of FIG. 2A.

FIG. 2C provides the elution results from a direct connect Protein A affinity column-ion exchange column of the same NIST mAb in buffer sample as used in the elution of FIG. 2A and FIG. 2B.

FIG. 3A provides the results taken from a direct connect Protein A affinity column-ion exchange column after a first injection of non-transfected CHO media with no mAb sample, running with 20 mM MES at pH 6.7 at 0.6 mL/min for 15 minutes.

FIG. 3B provides the results taken after a second injection on the direct connect Protein A affinity column-ion exchange column of FIG. 3A. The second injection utilizes an elution solution of 100 mM citric acid for elution from the Protein A column followed by a salt gradient.

FIG. 4A provides the elution results of a NISTmAb spiked into non-transfected CHO media sample from a Protein A column.

FIG. 4B provides the results taken after a first injection on a direct connect Protein A affinity column-ion exchange column when a sample of a NISTmAb spiked into non-transfected CHO media is used. The first injection comprises 20 mM MES at pH of 6.7 run at 0.6 mL/min for 15 minutes.

FIG. 4C provides the results taken after two injections on the direct connect Protein A affinity column-ion exchange column. The NISTmAB is eluted from the Protein A column with 2 microliters of 100 mM citric acid and ran with IEX salt gradient.

FIG. 5A provides the elution results of a mAb prepared in a CHO media sample from a Protein A column.

FIG. 5B provides the results taken after a first injection on an ion exchange column when a sample of a mAb prepared in CHO media is used. The buffer for the first injection is 20 mM MES at pH of 6.7 run at 0.6 mL/min for 15 minutes.

FIG. 5C provides the results taken after two injections on the direct connect Protein A affinity column-ion exchange column. The mAb is eluted from the Protein A column with 2 microliters of 100 mM citric acid and ran with IEX salt gradient.

### DETAILED DESCRIPTION

Disclosed herein are methods of performing tandem affinity chromatography and ion exchange chromatography (IEX) using a direct elution method from the affinity chromatography column to the IEX column (direct affinity-ion exchange chromatography; or alternatively direct affinity-IEX).

In conventional technologies, the use of different modes of separation (i.e., affinity chromatography and IEX chromatography) on a single sample requires the use of either valve switching or sample isolation from the first mode of separation (also known as a dimension). The former requires liquid chromatography systems with valve and trap configurations that can be used to first capture (‘trap’) an analyte of interest separated by a first dimension and divert its flow path to the second mode of separation. In these configurations, the modes of separation (dimensions) are decoupled—that is only the trapped analytes from the first dimension are transferred to and separated by the second dimension. Thus, the ability to separate multiple target analytes is limited, in part, by the system configuration and the efficiency of the valve/trap apparatuses. An alternative to valving is to first collect a sample fraction from a first mode of separation, and subsequently injecting the sample fraction onto the second mode of separation. This approach requires manual manipulation of small sample volumes, which can complicate workflows and introduce user error. These approaches are necessary as direct connection of one column type to another in the absence of a valve would result in either many peaks or single broad peaks being eluted from the first column into the second column, thereby reducing separation performance of the second column.

The direct affinity-IEX methods described herein circumvent the aforementioned issues with two-dimensional chromatography while permitting separation of a sample using two chromatographic modes of separation.

In particular, the affinity chromatography columns described herein result in a single load peak from the load step (i.e., a single peak of analytes, wherein the single peak of analytes is composed of one or more analytes that do not bind to the affinity column) and a single, elution peak from the elution step (i.e., a single peak of analytes, wherein the single peak of analytes is composed of one or more analytes), which is on the order of a standard HPLC/UHPLC injection (i.e., between 5-20 μL). Further, the affinity chromatography columns used in the instant technology have pressure compatibility with the IEX columns used herein. Thus, the peaks (the one or more analytes which elute together in either the load step or the elution step) that elute from the affinity chromatography column can be eluted directly onto the IEX column without the need for valving or fraction collection. In doing so, both the analytes from the loading step and the elution step are subsequently separated by the IEX column. The affinity chromatography step of a method described herein includes an affinity buffer solution, and the IEX chromatography step of a method described herein includes an IEX buffer.

Affinity chromatography and IEX chromatography are both adsorptive methods of chromatography (i.e., one or more target analytes are retained by the chromatography column until exposed to a buffer condition which causes the release of the one or more analytes). Therefore, methods of the present disclosure allow for a target analyte to be captured in the affinity chromatography column during a first affinity chromatography step and retained during a first IEX chromatography step. Then, during a second affinity chromatography step, the desired analyte may be eluted from the affinity chromatography column onto the IEX chromatography column, allowing the target analyte to be focused within the IEX chromatography column. A second IEX chromatography step may then be used to release the target one or more analytes from the IEX chromatography column.

Affinity-IEX chromatography methods disclosed herein are particularly well suited for elution with a gradient mobile phase (e.g., the transition between an affinity buffer and an IEX buffer) during an IEX elution step. One-dimensional affinity chromatography is commonly performed using a step gradient or slope gradient method. These methods typically result in broad peaks in the resulting chromatogram. These broad peaks are typically associated with volumes far larger than would be appropriate for IEX chromatography. By employing a gradient elution method in the IEX step, the affinity buffer used to elute an analyte may be gradually changed into an IEX buffer of increasing ionic strength, thereby allowing for the separation of analytes in the IEX column.

For example, over a period of time (e.g., from about 1 to 3 minutes) the mobile phase may be changed from the affinity buffer to the IEX buffer, Use of a gradient mobile phase forms a gradient of the IEX buffer that flows through the chromatography system (ranging from 0% IEX buffer (100% affinity buffer) to 100% of IEX buffer (100% affinity buffer).

### Affinity Chromatography Materials and Columns Thereof

The present technology utilizes affinity chromatography columns for the purification and isolation of a target analyte. The affinity chromatography columns are suitable for use in a high-performance liquid chromatography (HPLC) system or an ultra-high performance liquid chromatography (UHPLC) system and are designed for robust on-column affinity capture at the high pressures and flow conditions of said systems.

### Affinity Chromatography

A sample to be separated by a method disclosed herein may first be separated by an affinity chromatography column. Affinity chromatography serves to separate one or more components of a sample based on the strength of interaction between the component and an affinity agent included with the affinity chromatography column. The strength of these interactions can be controlled by, e.g., the pH of the mobile phase (i.e., the pH of the buffer). Affinity chromatography columns of the present disclosure are suitable for use in a high-performance liquid chromatography (HPLC) system or an ultra-high performance liquid chromatography (UHPLC) system and are designed for robust on-column affinity capture at the high pressures and flow conditions of said systems.

### Affinity Chromatography Materials

The affinity chromatography columns used in the methods disclosed herein include nonporous particles, which provide high surface area for conjugation of affinity agents and can withstand the pressures of HPLC and UHPLC systems. As such, in one aspect the affinity chromatography columns include a plurality of nonporous particles having an average particle size between 1.0 μm to 10 μm. In a preferred embodiment, the nonporous particles are nonporous polymer particles. In some embodiments, each particle within the plurality of particles is highly spherical with a smooth surface. In some embodiments, each particle within the plurality of particles is highly spherical with a bumpy, convex surface. Such materials have surface areas (measured in m²/g) that are close to their theoretical values. The theoretical surface area for a nonporous smooth sphere is equal to 6/{particle diameter×particle density}. For example, 1 μm polymer particles with a density of approximately 1 g/mL has a theoretical surface area of 6 m²/g, a 3.5 μm polymer particle with the same density has a theoretical surface area of 1.7 m²/g, and a 7 μm polymer particle with same density has a theoretical surface area of 0.9 m²/g.

The particles for use in the methods described herein are nonporous. While some pores or porosity may be incorporated within the particles as discontinuities or as microporosity, nonporous particles are those particles having a pore volume that is less than 0.1 cc/g of the material forming the particle. Preferably, nonporous particles have a pore volume that is less than 0.10 cc/g (e.g., 0.05 cc/g), and preferably less than 0.02 cc/g, in some embodiments. Pore volume is determined using methods known in the art based on multipoint nitrogen sorption experiments (Micromeritics ASAP 2400; Micromeritics Instruments Inc., Norcross, GA). Without wishing to be bound by theory, it is believed that the use of nonporous particles is advantageous as it removes diffusion of analytes into pores of the particles, thereby by improving kinetics of the binding and eluting steps of affinity chromatography.

The nonporous particles described herein have an average particle size of between 1 to 10 μm. In some embodiments, the particle size is about 1.7 μm. In some embodiments, the particle size is about 3.5 μm. In some embodiments, the particle size is about 7 μm.

The size (e.g., less than 10 microns), shape (e.g., spherical), and surface area (e.g., nonporous smooth or nonporous bumpy convex) create a form factor useful for affinity chromatography and affinity chromatography columns used in conjunction with HPLC and UHPLC systems. These systems operate under high pressures (e.g., typically greater than 3,000 psi, such as, for example, 5,000 psi, 10,000 psi, 12,000 psi, 15,000 psi and so forth). Therefore, the particles used herein are rigid particles such that the form factor is retained under HPLC and UHPLC operating conditions.

As used herein, the term “rigid particle,” as used herein, refers to the strength of the particle to withstand applied pressures under flow conditions. A rigid particle appears visually undamaged (i.e., maintains the same form factor without breaking, crushing, or alteration) in a scanning electron microscope image after exposure to pressures of 3,500 psi, wherein less than 10% of the observed particles are visually damaged. In addition, particles in a packed bed that are broken or deformed result in reduced flow and increased pressure as one would predict using the Kozeny-Carmen equation. Broken or deformed particles in a packed bed can increase pressure beyond levels suitable for use in HPLC or UHPLC.

Materials that meet the form factor requirements for forming a core (e.g., center or base) of the particles used herein include polymers, in particular organic polymers. Thus, in some embodiments, the nonporous particles include a nonporous polymer core. In some embodiments, the nonporous polymer core is divinylbenzene (DVB), for example divinylbenzene 80%. In some embodiments, the nonporous polymer core is formed to include two or more polymers. For example, in some embodiments the nonporous polymer cores include both DVB and polystyrene. In certain embodiments, the nonporous polymer cores can be manufactured to include a gradient in the polymer composition. For example, the inner portion of the core can be formed of 100% of first polymer (i.e., polymer A) and an outer portion of the core can be formed of 100% or some percentage greater than 0% of a second polymer (i.e., polymer B). Radially from the inner portion to the outer portion of the core, the percentage of polymer A and polymer B can vary to form the gradient in polymer composition. Other embodiments of nonporous polymer cores and particles suitable for use with the present technology are described in U.S. Patent Publication No. 2019/0322783, incorporated herein by reference.

Other nonporous materials can be utilized as long as the form factor of the particles can be maintained under the operating conditions of HPLC or UHPLC. That is, other materials, such as silica, metal oxides, hybrid inorganic-organic materials, or combinations thereof may be used to create nonporous spherical particles having an average particle size of less than 10 microns and the rigidity to retain form factor under high operating pressures (e.g., greater than 3000 psi).

### Affinity Agents

To form particles useful for affinity chromatography, the outer surface of the nonporous particle is conjugated, either directly or indirectly, to an affinity agent.

In embodiments for direct conjugation, the outer surface of the nonporous polymer core includes a hydrophilic surface, such as, for example, an epoxide. One or more affinity agents can be directly conjugated to the hydrophilic surface. The hydrophilic surface (or hydrophilic layer, used interchangeably herein) can be formed of a polymer, molecule or siloxane that has a high density of hydrophilic groups (e.g., hydroxyls, PEG, sugars or carbohydrates). The immobilization of these hydrophilic groups can occur by condensation (ester, amid, silanol, sily ether), polymerization (methacrylates, acrylates, styryl) epoxy activation (epihydrochlorin), or ether formation (direct attachment of PEG or carbohydrate groups by ether formation). Further examples include (3-glycidyloxypropyl)trimethoxysilane, (3-glycidyloxypropyl)triethoxysilane, polyacrylate, poly(methyl acrylate), and combinations thereof. Additionally or alternatively, these may include glycidol, glyceroltriglycidyl ether, and combinations thereof.

An affinity agent can be directly conjugated to the hydrophilic surface of the nonporous particle via linkers and methods known in the art, and described in Hermanson G, “Bioconjugate Techniques” 3^{rd} Edition, July 2013).

In embodiments for indirect conjugation, one or more streptavidin molecules are first directly conjugated to the hydrophilic surface of the nonporous particles as described above. Due to the strong affinity between biotin and streptavidin, the streptavidin molecules provide a binding site for biotinylated affinity agents, providing a functionalized particle with a specific affinity (based on the affinity of the affinity agent).

Various affinity agents are suitable for use in the disclosed methods. These include immunoglobulin-binding proteins, antibodies or antigen-binding fragments thereof, oligonucleotides and nucleic acids, or other ligand-binding proteins or peptides. In embodiments wherein the affinity agent is indirectly conjugated to the particle, the affinity agent must be biotinylated.

### Immunoglobulin-Binding Affinity Agents

In one aspect, the affinity agent is an immunoglobulin-binding protein. The immunoglobulin-binding protein provides accessible binding sites for an immunoglobulin, i.e., an antibody, provided that said antibody includes a conserved region that binds to the immunoglobulin-binding protein. In one embodiment, the immunoglobulin-binding protein is Protein A. In another embodiment, the immunoglobulin-binding protein is Protein G. In other embodiments, the immunoglobulin-binding protein is Protein A/G or Protein L. In some embodiments, the immunoglobulin-binding protein is directly conjugated to the surface of the nonporous particle. In some embodiments, the immunoglobulin-binding protein is indirectly conjugated to the surface of the nonporous particle. In embodiments wherein the immunoglobulin-binding protein is indirectly conjugated to the surface of the nonporous particle, the immunoglobulin-binding protein is a biotinylated immunoglobulin-binding protein. For example, a biotinylated Protein A, a biotinylated Protein G, a biotinylated Protein A/G, or a biotinylated Protein L.

Most immunoglobulins (Ig) consist of four polypeptide chains: two identical heavy chains and two identical light chains that are connected by disulfide bonds. Within a given heavy chain or light chain, there is both a variable and a constant region. The constant region, which includes 2-4 constant domains (depending on isotype), is highly conserved within a given isotype. As such, immunoglobulin-binding proteins that bind to a portion of the constant region are suitable for affinity capture of antibodies independent of the antibody's target antigen.

Immunoglobulin-binding proteins suitable for use in the present technology may exhibit strong binding affinity to the Fc portion of an antibody. This binding affinity can vary in strength by both isotype and species. For example, Protein A exhibits strong binding affinity to IgG isotypes but variable to no binding affinity to IgA, IgD, IgE, and IgM isotypes. Even within the IgG isotype, different subclasses can exhibit varied binding affinity. Protein A has high binding affinity to human IgG1, IgG2, and IgG4, but very weak binding affinity to IgG3. By contrast, Protein A binds to murine IgG3 but not to IgG1. Other examples of immunoglobulin-binding proteins, such as Protein G, have high binding affinity to all four subclasses of IgG. Methods for characterizing protein-protein interactions, including binding affinities across a range of environmental conditions, are well known in the art.

### Antibody Affinity Agents

In one aspect, the affinity agent is an antibody or antigen-binding fragment thereof. In some embodiments, the antibody or antigen-binding fragment thereof is a polyclonal antibody, a monoclonal antibody, a single-chain variable fragment (scFv), a nanobody, a monobody, a single domain antibody, a bispecific antibody, or a camelid. In some embodiments, the antibody or antigen-binding fragment thereof is an IgG, IgM, IgA, IgE, or IgD isotype. The antibody or antigen-binding fragment thereof may be derived from a human, mouse, rabbit, goat, or other species. In some embodiments, the antibody is a humanized antibody. In yet other embodiments, the antibody or antigen-binding fragment thereof is a biotinylated antibody or antigen-binding fragment thereof. That is, the biotinylated antibody or antigen-binding fragment thereof is a biotinylated polyclonal antibody, a biotinylated monoclonal antibody, a biotinylated scFv, a biotinylated nanobody, a biotinylated monobody, a biotinylated single domain antibody, a biotinylated bispecific antibody, or a biotinylated camelid.

In some embodiments, the affinity agent is an antibody or antigen-binding fragment thereof that specifically binds to insulin. In some embodiments, the antibody or antigen-binding fragment there of that specifically binds to insulin is a biotinylated antibody or antigen-binding fragment thereof. In some embodiments, the affinity agent is an antibody or antigen-binding fragment thereof that specifically binds to AAV9. In some embodiments, the antibody or antigen-binding fragment there of that specifically binds to AAV9 is a biotinylated antibody or antigen-binding fragment thereof. In some embodiments, the affinity agent is an antibody or antigen-binding fragment thereof that specifically binds to AAV2. In some embodiments, the antibody or antigen-binding fragment thereof that specifically binds to AAV2 is a biotinylated antibody or antigen-binding fragment thereof. In some embodiments, the affinity agent is an antibody or antigen-binding fragment thereof that specifically binds to an AAV capsid, such as AAV1, AAV2, AAV3, AAV4, AAV5, AAV6, AAV7, AAV8, and/or AAV9. In some embodiments, the antibody or antigen-binding fragment thereof that specifically binds to an AAV capsid is a biotinylated antibody or antigen-binding fragment thereof.

In some embodiments, the affinity agent is an antibody or antigen-binding fragment thereof that specifically binds to tacrolimus. In some embodiments, the antibody or antigen-binding fragment thereof that specifically binds to tacrolimus is a biotinylated antibody or antigen-binding fragment thereof. In some embodiments, the affinity agent is an antibody or antigen-binding fragment thereof that specifically binds to troponin. In some embodiments, the antibody or antigen-binding fragment thereof that specifically binds to troponin is a biotinylated antibody or antigen-binding fragment thereof. In some embodiments, the affinity agent is an antibody or antigen-binding fragment thereof that specifically binds to IgG. In some embodiments, the antibody or antigen-binding fragment thereof that specifically binds to IgG is a biotinylated antibody or antigen-binding fragment thereof. In some embodiments, the affinity agent is an antibody or antigen-binding fragment thereof that specifically binds to a cytokine. In some embodiments, the antibody or antigen-binding fragment thereof that specifically binds to a cytokine is a biotinylated antibody or antigen-binding fragment thereof. In some embodiments, the affinity agent is an antibody or antigen-binding fragment thereof that specifically binds to perfluoroalkyl substances (PFAS). In some embodiments, the antibody or antigen-binding fragment there of that specifically binds to PFAS is a biotinylated antibody or antigen-binding fragment thereof. In some embodiments, the affinity agent is an antibody or antigen-binding fragment thereof that specifically binds to a host cell protein (HCP). In some embodiments, the antibody or antigen-binding fragment thereof that specifically binds to a HCP is a biotinylated antibody or antigen-binding fragment thereof.

As used herein, the term “host cell protein” refers to process-related proteinaceous impurities present in a host cell culture or host cell line used during biopharmaceutical manufacturing and production.

### Oligonucleotide and Nucleic Acid Affinity Agents

In another aspect, the affinity agent is an oligonucleotide, nucleic acid, or oligomer. In some embodiments, the oligonucleotide can range from 5-50 nucleotides. In some embodiments, the nucleotide includes 25 nucleotides. Any or all of the nucleotides in a particular oligonucleotide or nucleic acid species can further be modified using methods known in the art, including biotinylating of the oligonucleotide or nucleic acid species. In particular, an oligonucleotide can be biotinylated on the 5′ or 3′ end.

Oligonucleotides suitable as affinity agents may be presented by the following Formula I:

B₀₋₁(Xₙ)ₚB′₀₋₁ (Formula I);
wherein B and B′ are independently a biotin group,
X is independently a nucleotide, nucleoside, or derivative thereof, including but not limited to adenosine, thymidine, guanosine, and cytidine;
n is independently 1-50; and
p is independently 1-50.

In some embodiments of Formula I, at least one of B or B′ is present (i.e., B or B′ is 1). In some embodiments, B is 1 and B′ is 0. In some embodiments, B is 0 and B′ is 1. In some embodiments, both B and B′ are 1. In some embodiments, both B and B′ are 0.

In some embodiments of Formula I, B is 1, X is thymidine, n is 25, p is 1, and B′ is 0. In some embodiments of Formula I, B is 0, X is thymidine, n is 25, p is 1, and B′ is 1. The resultant 5′-biotinylated or 3′-biotinylated oligonucleotide includes 25 thymidine units (i.e., a 25-mer of thymidine or dT₂₅).

In some embodiments, the affinity agent is a biotinylated oligonucleotide of Formula I. In some embodiments, the biotinylated oligonucleotide sequence is complementary to a target analyte sequence.

In one aspect, any nucleic acid-based affinity agent can be used, including biotinylated nucleic acid affinity agents and oligonucleotide affinity agents. The oligonucleotide may include deoxyribonucleic acids (DNA), ribonucleic acids (RNA), or a combination thereof. DNA oligonucleotides include the nucleotides cytidine, guanosine, adenosine, and thymidine. RNA oligonucleotides include the nucleotides cytidine, guanosine, adenosine, and uridine. In some embodiments, the oligonucleotide may include nucleic acid analogues (i.e., non-naturally occurring nucleic acids or analogues thereof). Examples of nucleic acid analogues include peptide nucleic acids, locked nucleic acids, glycol nucleic acids, threose nucleic acids, hexitol nucleic acids. Nucleic acid analogues are further reviewed in Wang et al., Molecules (2023) 28(20):7043. Oligonucleotides may further be modified at the nucleobase, sugar, or phosphodiester backbone with an array of chemical modifications which are further reviewed in Epple et al., Emerg. Top. Life. Sci. (2021) 5(5):691-697. In yet other embodiments, the nucleic acid affinity agent is an aptamer having specificity to a target analyte, including a biotinylated aptamer. Methods of biotinylating oligonucleotides, including modified oligonucleotides or oligonucleotides including non-naturally occurring nucleic acid analogues, are well known in the art and would be readily understood by a person of ordinary skill.

### Affinity Chromatography Column Body

The affinity chromatography materials, i.e., the nonporous particles conjugated to an affinity agent, are typically packed into a chromatographic column, thereby resulting in an affinity chromatography column. The column body is typically formed of a metal or a metal alloy, e.g., titanium or stainless steel.

In some embodiments, an alkylsilyl coating or other high-performance surface (HPS) is provided to limit or reduce non-specific binding of a sample with the walls or interior surfaces of the column body. Without wishing to be bound by any particular theory, it is believed than an alkylsilyl coating covering metal surfaces prevents or otherwise minimizes contact between fluids passing through the column. The alkylsilyl coating can be applied to the interior surfaces defining what is known as a wetted path of the column. A metal wetted path includes all surfaces formed from metal that are exposed to fluids during operation of the chromatographic column. The metal wetted path includes not only the column body walls but also metal frits disposed within the column. The coating may be applied not only to the wall of the column body but also to the frits.

In general, the alkylsilyl coating is applied through a vapor deposition technique. Vaporized precursors are charged into a reactor in which the part to be coated is located. These vaporized precursors react on the surfaces of the part to be coated to form a first layer of deposited material. The vapor deposition can be applied in a stepwise function to apply a number of layers of deposited material to the surfaces to grow a thickness of the coating and/or to apply layers of different materials (e.g., alternating between a first and second material) to form the coating.

In some embodiments, the alkylsilyl coating is applied to other portions of the liquid chromatography system. For example, the alkylsilyl coating can be applied to metal components residing upstream and downstream of the column. Specifically, the alkylsilyl coating can be applied to an injector of the liquid chromatography system and to post column tubing and connectors (e.g., tubing and connectors leading from the column to downstream components such as detectors). Further, the affinity chromatographic columns of the present technology do not require the addition of additional organic modifiers to reduce non-specific binding. Typically, the addition of an organic modifier (e.g., acetonitrile) may be necessary with sorbents used in affinity chromatography to reduce non-specific binding. Due to the already low non-specific binding of the columns of the present technology, no organic modifier is necessary.

In one embodiment, the alkylsilyl coating includes a hydrophilic, non-ionic layer of polyethylene glycol silane. In another embodiment, the alkylsilyl coating is formed from one or more of the following precursor materials bis(trichlorosilyl)ethane or bis(trimethoxysilyl)ethane. Other embodiments of alkylsilyl coatings suitable for use with the present technology are described in US Patent Publication No. 2019/0086371 and US Application Publication No. 2022/0118443.

### Affinity Buffers

A number of buffers are suitable for use with the methods provided herein. Affinity buffers suitable for use in liquid chromatography are known in the art and could be determined by a person of ordinary skill in the art. In some embodiments, the wash buffer has a pH of between 6.0 to 8.0. In some embodiments, the wash buffer has a pH of between 6.0 to 6.5, 6.5 to 7.0, 7.0 to 7.5, or 7.5 to 8.0. In some embodiments, the affinity buffer includes sodium phosphate, ammonium acetate, or ammonium formate. For example, but not by way of limitation, a suitable wash buffer may be 50-100 mM sodium phosphate, pH 7.2 to 7.4 or 100-200 mM ammonium formate pH 6.5. Additional washing buffers and concentrations thereof are described in the examples below.

In some embodiments, the wash buffer further includes an organic solvent. Examples of organic solvents suitable for use include acetonitrile and ethanol. In some embodiments, the concentration of the organic solvent is between 0-10% or any number between said range.

### Ion Exchange Chromatography

Following separation by the affinity chromatography column, any components of the sample that are eluted from the affinity chromatography column (i.e., any components which do not bind to the affinity chromatography column, or any components which elute from the affinity chromatography column in the presence of the affinity buffer) are directly eluted onto an ion exchange chromatography (IEX) column. In IEX chromatography, samples are separated based on their ionic interactions with oppositely charged moieties present on an ion exchange chromatography material (IEX chromatography material). The strength of these interactions can be controlled by, e.g., the pH and/or the ionic strength of mobile phase (i.e., the pH and/or the ionic strength of a buffer).

IEX chromatography includes, but is not limited to, cation exchange chromatography (CX), anion exchange chromatography (AEX), weak cation exchange chromatography (WCX), strong cation exchange chromatography (SCX), weak anion exchange chromatography (WAX), cationic (CEX), and strong anion exchange chromatography (SAX)

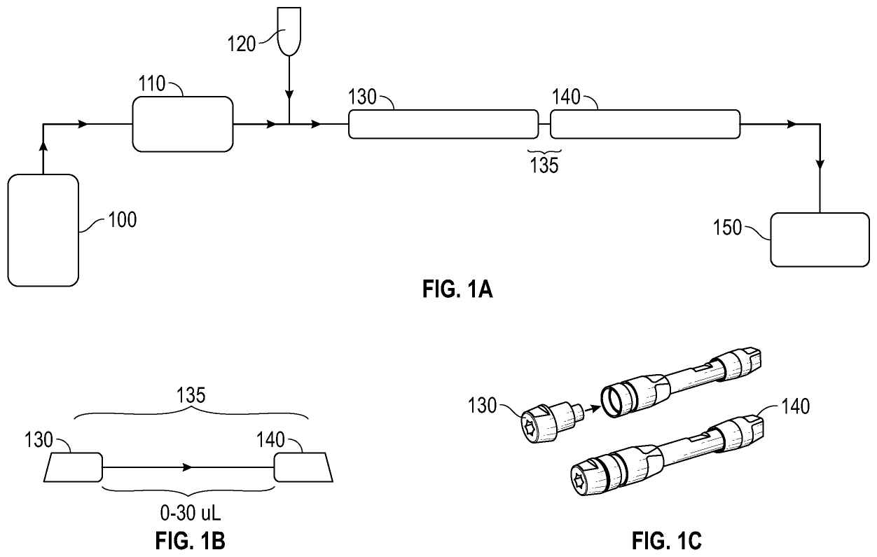

IEX chromatography columns of the present disclosure are in direct fluidic connection with the affinity chromatography column and downstream of the affinity chromatography column (see FIGS. 1A-1B). The IEX columns are suitable for use in a high-performance liquid chromatography (HPLC) system or an ultra-high performance liquid chromatography (UHPLC) system and are designed for robust on-column affinity capture at the high pressures and flow conditions of said systems.

### Ion Exchange Chromatography Materials

A number of materials are suitable for IEX columns used in conjunction with the present technology, provided that the IEX materials can withstand the high pressures associated with HPLC or UHPLC systems. For example, materials suitable for IEX column chromatography materials may be able to withstand a pressure of at least 3000 psi (e.g., at least 5000 psi, at least 10000 psi, at least 12000 psi, at least 15000 psi, etc.).

IEX chromatography materials may include a plurality of particles (e.g., porous particles, non-porous particles, or superficially porous particles). In some embodiments, the plurality of IEX particles have a particle size of between 1 μm to 10 μm (e.g., 3.5 μm, 4 μm, 5 μm, 6 μm, 7 μm, 8 μm, 9 μm, or 10 μm). In some embodiments, the plurality of IEX particles have a particle size of 1.7 μm. In some embodiments, the plurality of IEX particles have a particle size of 2.5 μm. In some embodiments, the plurality of IEX particles have a particle size of 3 μm. In some embodiments, the plurality of IEX particles materials have a particle size of 7 μm.

In some embodiments, the plurality of IEX particles are porous, non-porous, or superficially porous. In embodiments wherein the plurality of IEX particles are porous, the plurality of IEX particles have a pore size ranging from 125 Å to 450 Å. In some embodiments, the plurality of IEX particles s have a pore size of 125 Å, 200 Å, 250 Å, 300 Å, 400 Å, or 450 Å. In some embodiments, the plurality of IEX particles materials have a pore size of 250 Å.

The plurality of IEX particles may include, e.g., an inorganic polymer (e.g., silica), an organic polymer (e.g., a polymer formed from DVB), or a hybrid organic-inorganic polymer. The surface of each of the plurality of IEX particles is charged, thereby enabling electrostatic interactions between the IEX particle and the sample. For example, the surface of an IEX particle may include a sulfate group (providing a negatively charged surface) or an ammonium group (providing a positively charged surface. Exemplary materials which may form an IEX material of the present disclosure include those disclosed in, e.g., U.S. Pat. Nos. 11,433,320; 11,577,179; and 11,642,653; and U.S. Patent Publication Nos. US 20200284771 and US 20240425844 the materials for ion exchange chromatography of which are incorporated herein by reference.

### Ion Exchange Chromatography Column Body

The plurality of IEX particles is typically packed into a chromatographic column, thereby resulting in an IEX column. The column body is typically formed of a metal or a metal alloy, e.g., titanium or stainless steel.

In some embodiments, an alkylsilyl coating or other high-performance surface (HPS) is provided to limit or reduce non-specific binding of a sample with the walls or interior surfaces of the column body. Without wishing to be bound by any particular theory, it is believed than an alkylsilyl coating covering metal surfaces prevents or otherwise minimizes contact between fluids passing through the column. The alkylsilyl coating can be applied to the interior surfaces defining what is known as a wetted path of the column. A metal wetted path includes all surfaces formed from metal that are exposed to fluids during operation of the chromatographic column. The metal wetted path includes not only the column body walls but also metal frits disposed within the column. The coating may be applied not only to the wall of the column body but also to the frits.

In general, the alkylsilyl coating is applied through a vapor deposition technique. Vaporized precursors are charged into a reactor in which the part to be coated is located. These vaporized precursors react on the surfaces of the part to be coated to form a first layer of deposited material. The vapor deposition can be applied in a stepwise function to apply a number of layers of deposited material to the surfaces to grow a thickness of the coating and/or to apply layers of different materials (e.g., alternating between a first and second material) to form the coating.

In some embodiments, the alkylsilyl coating is applied to other portions of the liquid chromatography system. For example, the alkylsilyl coating can be applied to metal components residing upstream and downstream of the column. Specifically, the alkylsilyl coating can be applied to an injector of the liquid chromatography system and to post column tubing and connectors (e.g., tubing and connectors leading from the column to downstream components such as detectors). Further, the affinity chromatographic columns of the present technology do not require the addition of additional organic modifiers to reduce non-specific binding. Typically, the addition of an organic modifier (e.g., acetonitrile) may be necessary with sorbents used in affinity chromatography to reduce non-specific binding. Due to the already low non-specific binding of the columns of the present technology, no organic modifier is necessary.

In one embodiment, the alkylsilyl coating includes a hydrophilic, non-ionic layer of polyethylene glycol silane. In another embodiment, the alkylsilyl coating is formed from one or more of the following precursor materials bis(trichlorosilyl)ethane or bis(trimethoxysilyl)ethane. Other embodiments of alkylsilyl coatings suitable for use with the present technology are described in US Patent Publication No. 2019/0086371 and US Application Publication No. 2022/0118443.

In some embodiments, the IEX column body includes a length of from 50 mm to about 500 mm (e.g., 50 mm, 100 mm, 150 mm, 200 mm, 250 mm, 300 mm, 350 mm, 400 mm, 450 mm, 500 mm, etc.). In some embodiments, the IEX column body includes an internal diameter of from 1 mm to 10 mm (e.g., 2.1 mm, 4.6 mm, etc). In some embodiments, the IEX column body include a length of 50 mm and an internal diameter of 2.1 mm. In some embodiments, the IEX column body includes a length of 100 mm and an internal diameter of 2.1 mm.

### Ion Exchange Buffer

IEX buffers suitable for use in the methods described herein may include an ammonium cation (e.g., ammonium formate, ammonium acetate, or ammonium bicarbonate). In some embodiments, the IEX buffer includes ammonium acetate. The IEX buffer may be characterized by a pH of from 4 to 9 (e.g., 4, 5, 6, 7, 8, or 9). In some embodiments, the IEX buffer is an MS-compatible buffer. In some embodiments, the elution buffer is a MALS-compatible buffer.

Critically, IEX buffers of the present invention do not cause denaturation or degradation of either components of a sample bound to the affinity chromatography column or the affinity agent of the affinity chromatography column. In some embodiments, any components retained by the affinity chromatography column are not released by the IEX buffer. For example, after a chromatography system of the present disclosure is treated with a first affinity buffer, a first IEX buffer may be used which does not cause the release or denaturation of any component still bound to the affinity chromatography column. In some embodiments, one or more components retained by the affinity chromatography column are released by the IEX buffer without causing degradation or denaturation of either a retained component or an affinity ligand. For example, after a chromatography system of the present disclosure is treated with a first affinity buffer and a first IEX buffer (which does not cause the release of one or more components retained by the affinity chromatography column), a second IEX buffer may be used, wherein the second IEX buffer is configured to release one or more retained components from the affinity chromatography column, and also separate any eluted components in the IEX chromatography column.

Exemplary IEX buffers include those disclosed in e.g., U.S. Pat. Nos. 11,433,320; 11,577,179; and 11,642,653; and U.S. Patent Publication Nos. US 20202284771 and US20240425844; the mobile phases and buffers of which are incorporated herein by reference.

### Zero and Low Volume Unions

The present technology and methods utilize an affinity chromatography column and IEX column in direct fluidic connection. The direct fluidic connection can be achieved via a zero dead volume or low volume union. The zero and/or low volume union eliminates the need for the use of a valve switch or sample loop that are typically required to achieve separation of a sample by more than one chromatographic mode of separation.

A zero dead volume union introduces no volume (i.e., about 1 μL or less of volume) to the flow path. A low volume union introduces minimal volume (i.e., between about 0 to about 30 μL of volume) to the flow path.

Zero dead volume unions (also referred to as connectors) and low volume unions (also referred to as connectors) are known in the art. Examples of said unions include the VanGuard™ FIT (available from Waters Technologies Corporation, Milford MA) and as described in U.S. Pat. Nos. 8,449,769 and 9,724,621, incorporated herein by reference.

Additional examples include, but are not limited to, the Sulfinert Zero Dead Volume Union (available from Shimadzu), the Idex High-Pressure Zero Dead Volume Union (available from Fisher Scientific), and the Swagelok® Zero Dead Volume Union (available from Millipore Sigma).

Zero dead volume and low volume unions include male-male unions, female-female unions, and male-female unions, and can be used to connect the affinity chromatography column directly to the IEX column.

### Chromatography Systems

The chromatography systems described herein include an affinity chromatography column in direct fluidic connection to a IEX column. The direct fluidic connection between the affinity chromatography column and the IEX column is via a zero dead volume or low volume union. The chromatography system may further include a solvent pump, solvent mixer, a sample injector, and one or more detectors.

FIGS. 1A-1C depict an exemplary liquid chromatography (LC) system of the present technology. FIG. 1A demonstrates the LC system, with the flow path represented by the arrows. Solvent pump (100) pumps the solvent to the solvent mixer (110). A sample, which includes the target analyte, is loaded into the system using a sample injector (120) and flowed through the affinity chromatography column (130). The target analyte is eluted from the affinity chromatography column (130) directly onto the IEX column (140). Affinity chromatography column (130) and IEX column (140) are in direct fluidic connection via a zero dead volume or low volume union (135). Once eluted from the IEX column, the sample can be analyzed via a detector (150). For example, while FIG. 1A depicts a single detector (150), it is understood that the single detector (150) may be absent, or there may be multiple detectors connected in fluidic series.

FIG. 1B provides an enlarged view of the direct fluidic connection between the affinity chromatography column (130) and the IEX column (140). The flow path of 135 is between 0-30 uL (microliters) of volume. In some embodiments, the flow path of 135 is less than 1 μL.

FIG. 1C depicts an exemplary union between the affinity chromatography column (130) and the IEX column (140).

The chromatography systems described herein allow for direct affinity-IEX in the absence of valve switching components. Accordingly, the present disclosure features chromatography systems without a valve switch along the flow path of the chromatography system. The chromatography systems described herein further allow for direct affinity-IEX in the absence of trap configurations. Accordingly, in some embodiments, a trap is absent from the flow path of the chromatography system.

In some embodiments, the size IEX column (140) is connected in fluidic series to one or more detectors. Detectors suitable for use in the methods disclosed herein include detectors for ultraviolet spectroscopy, fluorescence spectroscopy, mass spectrometry, and/or other detectors used for the characterization of analytes, such as, for example, multi-angle light scattering (MALS) detectors and charged aerosol detectors (CAD). In some embodiments, the liquid chromatography system is connected in series to a detector for ultraviolet spectroscopy. In some embodiments, the liquid chromatography system is connected in series to a detector for fluorescence spectroscopy. In some embodiments, the liquid chromatography system is connected in series to detector for mass spectrometry. In some embodiments, the liquid chromatography system is connected in series to a MALS detector. In some embodiments, the liquid chromatography system is connected in series to a CAD. In some embodiments, the liquid chromatography system is connected to one or more of the detectors in series.

### Methods of Use

The methods disclosed herein pertain to direct affinity-IEX for the purification and characterization of a target analyte from a sample. The method includes loading a sample including the target analyte onto an affinity chromatography column, washing the affinity chromatography column with an affinity buffer, eluting the target analyte from the affinity chromatography column directly onto the IEX column, and eluting the target analyte from the IEX column. In embodiments wherein the IEX column is connected in fluidic series to one or more detectors, the methods further include detecting the target analyte using the one or more detectors (e.g., a UV detector, a fluorescence detector, a MALS detector, and/or an MS detector). As such, the direct affinity-IEX methods described herein afford the analysis of a sample across two modes of separation, including the analysis of peaks that do not bind to the affinity column (i.e., flow through the column in the presence of the first affinity buffer, i.e. “load peaks”) and peaks that elute from the affinity column in the presence of the second affinity buffer (i.e., “elution peaks”).

Methods of the present disclosure may employ one or more buffers (e.g., affinity buffers, IEX buffers) during a step of a method. An affinity buffer may be employed during an affinity chromatography step to control the binding of one or more analytes to the affinity chromatography column. For example, a first affinity buffer may be employed to separate a collection of analytes with affinity for the chromatography column from a collection of analytes without affinity for the affinity chromatography column. A second affinity buffer may later be used to reduce the affinity of the analytes to the affinity chromatography column, thereby allowing the analytes to be eluted from the affinity chromatography column to the IEX column. An IEX buffer may be employed during an IEX chromatography step to control the binding of one or more analytes to the IEX chromatography column.

Each buffer may contain one or more specific components to aid in the performance of a method step. For example, an affinity buffer may include an acid (e.g., phosphoric acid) at a concentration sufficient to modify the affinity of an analyte to an affinity chromatography column. As a further example, an IEX buffer may include a salt (e.g., sodium chloride) at a concentration sufficient to modify the affinity of an analyte to an affinity chromatography column.

In some embodiments, the skilled artisan may select second affinity buffer or a second IEX buffer such that one or more analytes remain associated with the affinity chromatography column or the IEX chromatography column after the elution step (e.g., an analyte with sufficient affinity for the affinity chromatography column that it was not released by the first affinity buffer or the second affinity buffer; and/or an analyte with sufficient affinity for the IEX chromatography column that it was not released by the first IEX buffer or the second IEX buffer). These retained analytes may be eluted from the chromatography column by repeating the steps of injecting an additional affinity buffer and/or injecting an additional IEX buffer an appropriate number of times. In some embodiments, each subsequent affinity buffer may be configured to reduce the binding affinity of the analyte to the affinity chromatography column. For example, each affinity buffer may include an increased concentration of acid (e.g., an increased concentration of phosphoric acid); or each IEX buffer may include an increased ionic strength (e.g., an increased concentration of sodium chloride). In preferred embodiments, methods of the present disclosure include no more than two affinity buffers. In some embodiments, the second affinity buffer and the second IEX buffer may be the same or similar buffer compositions. This step may be repeated as many times as necessary to release the desired analyte from the chromatography system.

In some embodiments, one or more IEX elution steps may be performed for each affinity elution step. For example, an IEX elution step may be sufficient to elute a first analyte from the IEX chromatography column, but insufficient to remove one or more additional analytes from the chromatography column. In some embodiments, each IEX elution step may be performed with a unique IEX buffer (e.g., a first IEX buffer, a second IEX buffer, a third IEX buffer, etc.). Each successive IEX buffer may be characterized by an increasing ionic strength (e.g., an increasing concentration of sodium chloride), thereby improving the ability of each successive IEX elution step to remove an analyte from the IEX chromatography column. Each affinity elution step may include one or more IEX elution steps.

In some embodiments, the IEX buffer or IEX buffers used in an affinity elution step may be identical. For example, the first IEX buffer injected into the chromatography system after the first affinity elution step, and the first IEX buffer injected into the chromatography system after the second affinity elution step may be identical. In some embodiments, the IEX buffer or IEX buffers used in an affinity elution step may be unique.

An exemplary method of the present disclosure is now described. First, a sample is loaded onto the affinity chromatography column in the presence of a first affinity buffer. The affinity chromatography column is then washed with the first affinity buffer. Analytes with an affinity for the affinity chromatography column are retained by the affinity chromatography column, and analytes without affinity for the affinity chromatography column are eluted to the IEX column as the load peak described above. A first IEX buffer is then injected into the chromatography system. The IEX buffer passes through the affinity chromatography column and interacts with the one or more analytes of the load peak in the IEX chromatography column, thereby allowing elution and separation of the load peak based on affinity for the IEX chromatography column.

A second affinity buffer is then injected into the chromatography system. The second affinity buffer is configured to release one or more analytes bound to the affinity chromatography column in the presence of the first affinity buffer from the column, allowing the one or more analytes to flow through the affinity chromatography column onto the IEX chromatography column as the elution peak described above. A second IEX buffer is then injected into the chromatography system, interacting with the analytes of the elution peak in the IEX chromatography column, thereby allowing elution and separation of the elution peak based on affinity for the IEX chromatography column.

One or more target analytes can be eluted from the affinity chromatography column using any appropriate elution method. However, the elution method selected must be capable of releasing the analyte from the affinity chromatography column, while promoting the readsorption of the analyte by the IEX chromatography column. For this reason, preferred embodiments of the present disclosure utilize a gradient elution (i.e., a steady modulation in the concentration of an affinity buffer in the mobile phase). For example, the gradient may be from a mobile phase not including an affinity buffer, to a mobile phase including the affinity buffer. A gradient elution may then be used to modulate the composition of IEX buffer in the mobile phase.

As the flow path between the affinity chromatography column and the IEX column is uninterrupted, the buffers used in the provided methods are compatible with both columns. Thus, the methods provide for direct affinity-IEX methods without solvent mismatch or the need for buffer exchange between modes of separation. Further, as the buffers used for both columns are the same, the disclosed methods eliminate or otherwise minimize the need for column equilibration between samples.

### Advantages of Direct Connect Method

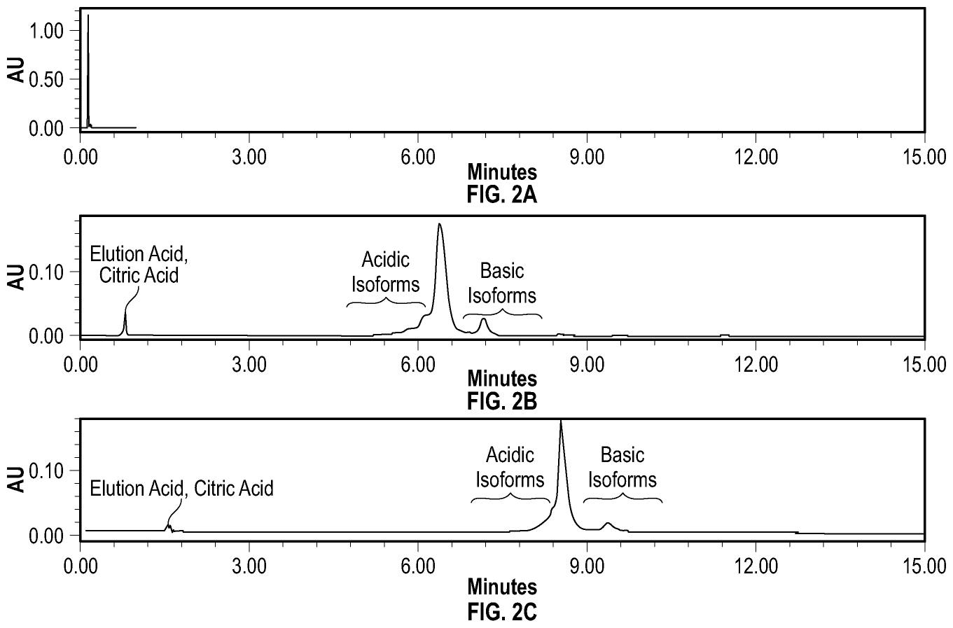

FIGS. 2A-2C illustrate the benefits of using the direct connect (i.e., direct Affinity to IEX Chromatography Columns) methods and devices of the present technology. In FIG. 2A, the results of a 2 microliter citric acid elution from a single Protein A column are provided. For this chromatographic result a sample of NISTmAb in buffer was loaded on a Protein A Column (i.e., BioResolve Protein column, 2.1×20 mm, 3 micron particles, available from Waters Corporation, Milford, MA). Prior to elution a 20 mM, 2-(N-morpholino)ethanesulfonic acid (MES) buffer with a pH 6.8 was used to condition the Protein A column (flow rate 0.6 mL/min, UV280, QSM, 80 pont/sec, runtime for 1 minute). To elute from the Protein A column two isocratic runs with 20 mM MES were used; injection 1: sample injection, injection 2, 2 microliters of 100 mM citric acid. The elution results provided in FIG. 2A illustrate that just a single peak was observed. From this peak only titer information can be measured.

FIG. 2B provides the results of the same sample as FIG. 2A, but using an IEX column (BioResolve SCX mAb column, 4.6×100 mm, 3 micron particles, available from Waters Corporation, Milford, MA). The chromatographic results shown in FIG. 2B are generated using a similar buffer to condition the column as was used in the Protein A column (20 mM MES buffer with pH 6.7). To elute a gradient using mobile phase A (20 mM MES pH 6.7) and mobile phase B (20 mM MES pH 6.7, 1M NaCl) was used according to the following gradient schedule 0 min 94% A, 0-2 min 94-92% A, 2 -12 min 92-89% A, 12-12.5 min 89% A, 12.5-15 min 99% A. The elution results provided in FIG. 2B illustrate that the IEX separation provides the charge states of the NISTmAb.

FIG. 2C provides results of the same sample (NISTmAb in buffer) as the results for FIG. 2A and FIG. 2B, however for FIG. 2C a direct connect ProA-IEX column and method was used. Specifically, the Protein A column used to capture the results of FIG. 2A was connected to the IEX column used to capture the results of FIG. 2B with a zero volume connector. The injection elution method used was as follows. Injection 1: inject sample, run 20 mM MES pH 6.7 at 0.6 mL/min for 15 min between both columns. Injection 2, inject elution acid: 2 microliters 100 mM citric acid, run IEX salt gradient with mobile phase A being 20 mM MES pH 6.7, mobile phase B being 20 mM MES pH 6.8, 1M NaCl. The gradient schedule was as follows: schedule 0 min 94% A, 0-2 min 94-92% A, 2 -12 min 92-89% A, 12-12.5 min 89% A, 12.5-15 min 99% A. The elution results shown in FIG. 2C illustrate the advantage of the direct connect method over the results shown in FIGS. 2A and 2B. That is, the direct connect method provides both titer and charge distribution data while maintaining resolution of the results. Moreover, no separate clean up step is needed for the method with results shown in FIG. 2C. That is, generally for IEX experiments, sample preparation is needed to clean up the samples. Sample preparation is time consuming and can lead to sample loss, which can create inaccuracies in titer information. One of the advantages of using the direct connect of the affinity to IEX column of the present technology, is elimination of a separate clean up step. The peak area difference observed between FIG. 2B and FIG. 2C is less than 5%, an thus the resolution of the information obtained in the method of the present technology (i.e., FIG. 2C) is maintained even though the sample clean up (i.e., sample preparation) is eliminated.

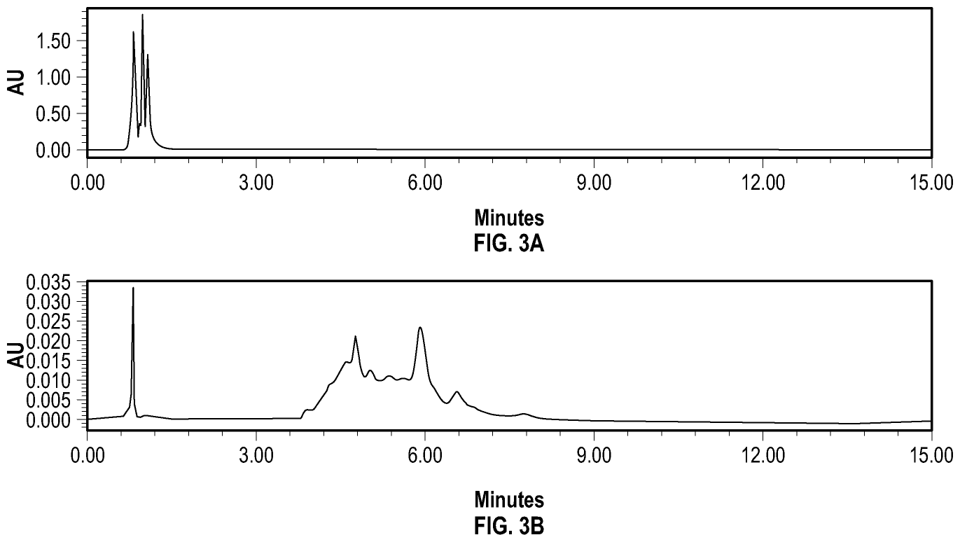

FIG. 3A and FIG. 3B illustrate the sensitivity of the direct connect method of the present technology. The chromatograms shown in FIG. 3A and FIG. 3B are produced using the direct connect Protein A column (BioResolve Protein column, 2.1×20 mm, 3 micron particles, available from Waters Corporation, Milford, MA) in direct contact with the IEX column (BioResolve SCX mAb column, 4.6×100 mm)of non-transfected CHO media with no monoclonal antibody sample. That is, FIG. 3A and FIG. 3B provide the results of running the elution on the media. Specifically, FIG. 3A provides the results after a first injection of buffer (20 mM MES); whereas FIG. 3B provides the results after a second injection, which includes 2 microliters of 100 mM of citric Acid followed by the IEX salt gradient with is mobile phase A being 20 mM MES pH 6.7, mobile phase B being 20 mM MES pH 6.8, 1M NaCl.

The sensitivity of the methods of the present technology is illustrated in that media components were separated and resolvable, which allows for separation of these media components from target sample components when a sample is used. As illustrated by the results of FIG. 3A and FIG. 3B, a majority of the media components are eluted after the first injection and are not retained by either the Protein A column or the IEX column. However, some media components are retained by the Protein A column and then are separated by the IEX column after the elution injection (FIG. 3B). As a result, information about the media is provided. This information on the media would not otherwise be possible from experiments that are run separately (i.e., separation of Protein A column from the IEX column). This information on the CHO media can then be utilized in the evaluation of impurities observed with mAb prepared with this specific CHO media.

### EXAMPLES

### Example 1: Exemplary Method of Using a Direct Affinity-IEX Chromatography System

The following example describes the characterization of an exemplary sample (e.g., an immunoglobulin sample, an antibody sample, an oligonucleotide sample, etc.) using a direct affinity-ion exchange (direct affinity-IEX) chromatography system. The affinity chromatography column is composed of a plurality of nonporous particles with an average size of from about 1 μm to about 10 μm. The plurality of nonporous particles includes an affinity agent (e.g., an immunoglobulin-binding affinity agent, an antibody affinity agent, or an oligonucleotide affinity agent). The ion exchange chromatography column is composed of a plurality of non-porous particles with an average particle size of from about 1 μm to about 10 μm. The plurality of non-porous particles includes a charged surface (e.g., a surface including a sulfate group or a surface including an ammonium group). The affinity chromatography column and the IEX column are joined by either a zero dead volume union or a low dead volume union.

The sample is loaded onto the affinity chromatography column, and the affinity chromatography column is washed with a first affinity buffer. Components of the sample with an affinity for the affinity chromatography column are retained by the affinity chromatography column, and analytes without affinity for the affinity chromatography column are eluted through the affinity column onto the IEX column. A first IEX buffer is then injected into the chromatography system. The first IEX buffer is configured to not interact with the components of the sample which remain bound to the affinity chromatography column (e.g., protein components of the sample are not denatured by the first IEX buffer, all components retained by the affinity chromatography column remain retained by the affinity chromatography column, etc.). After passing through the affinity chromatography column, the first IEX buffer interacts with the components of the sample which eluted from the affinity chromatography column onto the IEX column. These components of the sample are then separated by the IEX chromatography column. In order to better separate these components using IEX chromatography, a gradient mobile phase may be used.

After elution of the components in the IEX chromatography column is complete, a second affinity buffer is then injected into the chromatography system. The second affinity buffer is configured to release one or more of the components previously retained by the affinity chromatography column, allowing the one or more previously retained components to flow through the affinity chromatography column onto the IEX chromatography column. A second IEX buffer is then injected into the chromatography system. The second IEX buffer then interacts with the previously retained components in the IEX chromatography column, thereby allowing separation of the previously retained components by IEX chromatography. As with the first IEX buffer, a gradient mobile phase may be used to better separate the previously retained components.

It is contemplated that in some embodiments, the second affinity buffer and the second IEX buffer may be the same buffer. In other words, a buffer used to dissociate any retained analytes from an affinity chromatography column may further serve as the IEX buffer in the IEX step of the method.

### Example 2: Alternative Exemplary Method of Using a Direct Affinity-IEX Chromatography System

The following example describes the characterization of an exemplary sample (e.g., an immunoglobulin sample, an antibody sample, an oligonucleotide sample, etc.) using a direct affinity-ion exchange (direct affinity-IEX) chromatography system. The affinity chromatography column is composed of a plurality of nonporous particles with an average size of from about 1 μm to about 10 μm. The plurality of nonporous particles includes an affinity agent (e.g., an immunoglobulin-binding affinity agent, an antibody affinity agent, or an oligonucleotide affinity agent). The ion exchange chromatography column is composed of a plurality of non-porous particles with an average particle size of from about 1 μm to about 10 μm. The plurality of non-porous particles includes a charged surface (e.g., a surface including a sulfate group or a surface including an ammonium group). The affinity chromatography column and the IEX column are joined by either a zero dead volume union or a low dead volume union.

The sample is loaded onto the affinity chromatography column, and the affinity chromatography column is washed with a first affinity buffer. Components of the sample with an affinity for the affinity chromatography column are retained by the affinity chromatography column, and analytes without affinity for the affinity chromatography column are eluted through the affinity column onto the IEX column. A first IEX buffer is then injected into the chromatography system. The first IEX buffer is configured to not interact with the components of the sample which remain bound to the affinity chromatography column (e.g., protein components of the sample are not denatured by the first IEX buffer, all components retained by the affinity chromatography column remain retained by the affinity chromatography column, etc.). After passing through the affinity chromatography column, the first IEX buffer interacts with the components of the sample which eluted from the affinity chromatography column onto the IEX column. A subset of the components of the sample which eluted from the affinity chromatography column are retained by the IEX chromatography column, and the remaining components are then separated by IEX chromatography in the IEX chromatography column.

A second IEX buffer (e.g., an IEX buffer of a different ionic strength) is then applied to the chromatography system. Like the first IEX buffer, the second IEX buffer is configured to not interact with the components of the sample which remain bound to the affinity chromatography column. The second IEX buffer then releases one or more of the components of the sample previously retained by the IEX column, and separates them by IEX chromatography in the IEX chromatography column. This step is repeated using as many IEX buffers as necessary (e.g., a third IEX buffer, a fourth IEX buffer, a fifth IEX buffer, a sixth IEX buffer, etc.) to release all components of the sample retained by the IEX chromatography column.

After elution of the components in the IEX chromatography column is complete, a second affinity buffer is then injected into the chromatography system. The second affinity buffer is configured to release one or more of the components previously retained by the affinity chromatography column, allowing the one or more previously retained components to flow through the affinity chromatography column onto the IEX chromatography column. A first additional IEX buffer is then injected into the chromatography system. As with the first IEX buffer, the first additional IEX buffer is configured to not interact with any components still retained by the affinity chromatography column, and instead pass directly to the IEX chromatography column. A subset of the components of the sample which eluted from the affinity chromatography column are retained by the IEX chromatography column, and the remaining components are then separated by IEX chromatography in the IEX chromatography column.

A second additional IEX buffer (e.g., an IEX buffer of a different ionic strength) is then applied to the chromatography system. Like the first additional IEX buffer, the second additional IEX buffer is configured to not interact with the components of the sample which remain bound to the affinity chromatography column. The second additional IEX buffer then releases one or more of the components of the sample previously retained by the IEX column, and separates them by IEX chromatography in the IEX chromatography column. This step is repeated using as many additional IEX buffers as necessary (e.g., a third additional IEX buffer, a fourth additional IEX buffer, a fifth additional IEX buffer, a sixth additional IEX buffer, etc.) to release all components of the sample retained by the IEX chromatography column.

### Example 3: Detection of Analytes in Direct Affinity-IEX Chromatography

The following example describes methods of detecting one or more analytes present in a sample, such as the sample purified in Example 1 or Example 2 above. A detector (e.g., a UV detector, an MS detector, or a MALS detector) may be included in a chromatography system of the present disclosure. In order to ensure compatibility with the detector, the buffers used throughout Example 1 and Example 2 (i.e., all affinity buffers and all IEX buffers) are selected to also be compatible with the detector. For example, a UV compatible buffer is used in combination with a UV detector, an MS compatible buffer may be used in combination with an MS detector, and a MALS detector may be used in combination with a MALS detector.

The eluent obtained from Example 1 or Example 2 flows from the IEX chromatography column to the detector, and the detector performs an experiment (e.g., a UV experiment, an MS experiment, or a MALS experiment) to detect the presence of one or more analytes in the eluent.

### Example 4: Direct ProA-SCX Affinity-IEX Chromatography

The following example describes a method of performing direct affinity-IEX chromatography using a Protein A (ProA) affinity chromatography column and a strong cation exchange (SCX) chromatography column. The method may be particularly useful in the purification of monoclonal antibodies (mAbs).

A direct affinity-IEX chromatography system is formed by joining a ProA affinity chromatography column and an SCX chromatography column via either a zero dead volume union or a low dead volume union. A sample (including a target mAb) is loaded onto the affinity chromatography column, and the affinity chromatography column is washed with a first affinity buffer. For example, the first affinity buffer may be the BioResolve CX buffer (available from Waters Technology Corporation), the CX-1 buffer (available from ThermoFisher) or the IonHance CX-MS buffer (available from Waters Technology Corporation). One or more components from the sample pass through the affinity chromatography column and the IEX chromatography column while the target mAb is retained by the IEX chromatography column.

The target mAb is then eluted from the affinity chromatography column to the ion exchange chromatography column using an IEX buffer. The IEX buffer may include acid, thereby weaking the affinity of the target mAb for the affinity ligand on the affinity chromatography column. The target mAb then elutes from the IEX chromatography column in the presence of the IEX buffer.

The target mAb may then be collected and/or passed to a detector, such as a UV detector or an MS detector. The affinity buffer and the IEX buffer may be selected to be compatible with UV detection.

### Example 5: Direct AAV-AEX Affinity-IEX Chromatography Using Gradient Elution

The following example describes a method of performing direct affinity-IEX chromatography using an Adeno-Associated Virus (AAV) affinity chromatography column and w anion exchange (AEX) chromatography column. The method may be particularly useful in the purification of different AAVs.

A direct affinity-IEX chromatography system is formed by joining an AAV affinity chromatography column and an AEX chromatography column via either a zero dead volume union or a low dead volume union. A sample (including a target AAV) is loaded onto the affinity chromatography column, and the affinity chromatography column is washed with a first affinity buffer. Preferably, the first affinity buffer has a pH of from 8 to 9 and includes 20 mM ammonium bicarbonate, and 15 mM ammonium hydroxide. One or more components from the sample pass through the affinity chromatography column and the IEX chromatography column while the target AAV is retained by the IEX chromatography column.

The target AAV is then eluted from the affinity chromatography column to the ion exchange chromatography column using pH gradient to transition the mobile phase from the affinity buffer to an IEX buffer. For example, the IEX buffer may include 30 mM acetic acid and 15 mM formic acid. At time t=0 minutes, the mobile phase consisted of 40% IEX buffer and 60% affinity buffer. From time t=0.5 minutes to t=15.5 minutes, the mobile phase was gradually increased in IEX buffer until the mobile phase consisted of 80% IEX buffer and 20% affinity buffer at t=15.5 minutes. Then, from time t=15.5 minutes to t=15.6 minutes, the mobile phase was ramped to be composed of 100% IEX buffer at time t=15.6 minutes. The mobile phase was held at 100% IEX buffer until t=18 minutes, then reduced to 40% IEX buffer from time t=18 minutes to t=18.1 minutes. The mobile phase was then maintained at 40% IEX buffer until t=30 minutes.

The eluent from the IEX chromatography step was then detected using a UV detector set to operate at 260 nm and 280 nm.

### Example 6: Direct mRNA-AEX Affinity-IEX Chromatography

The following example describes a method of performing direct affinity-IEX chromatography using an mRNA affinity chromatography column and an anion exchange chromatography (AEX) column. Preferably, the present method utilizes a strong anion exchange (SAX) chromatography column or a WAX (weak anion exchange) chromatography column. The method may be particularly useful in the purification of nucleic acids.

A direct affinity-IEX chromatography system is formed by joining an mRNA affinity chromatography column and an AEX chromatography column via either a zero dead volume union or a low dead volume union. A sample (including a target oligonucleotide) is loaded onto the affinity chromatography column, and the affinity chromatography column is washed with a first affinity buffer. For example, the first affinity buffer include tris(hydroxymethyl)aminomethane (tris), triethylammonium acetate (TEAA), or bis(trispropane) at a pH of from 7.5 to 9. One or more components from the sample pass through the affinity chromatography column and the IEX chromatography column while the target nucleic is retained by the IEX chromatography column.

The target oligonucleotide is then eluted from the affinity chromatography column to the ion exchange chromatography column using an IEX buffer. The IEX buffer may include water at pH 7. The target oligonucleotide then elutes from the IEX chromatography column in the presence of the IEX buffer.

The target oligonucleotide may then be collected and/or passed to a detector, such as a UV detector or an MS detector. The affinity buffer and the IEX buffer may be selected to be compatible with UV detection.

### Example 7: Purifying and Characterizing mAbs in CHO Culture Media Using Direct ProA Affinity-IEX Chromatography

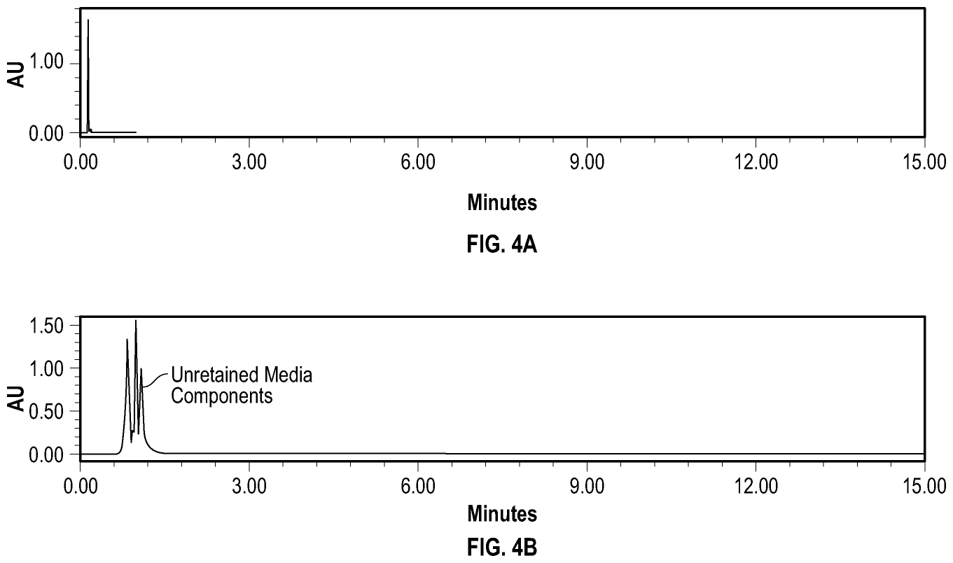

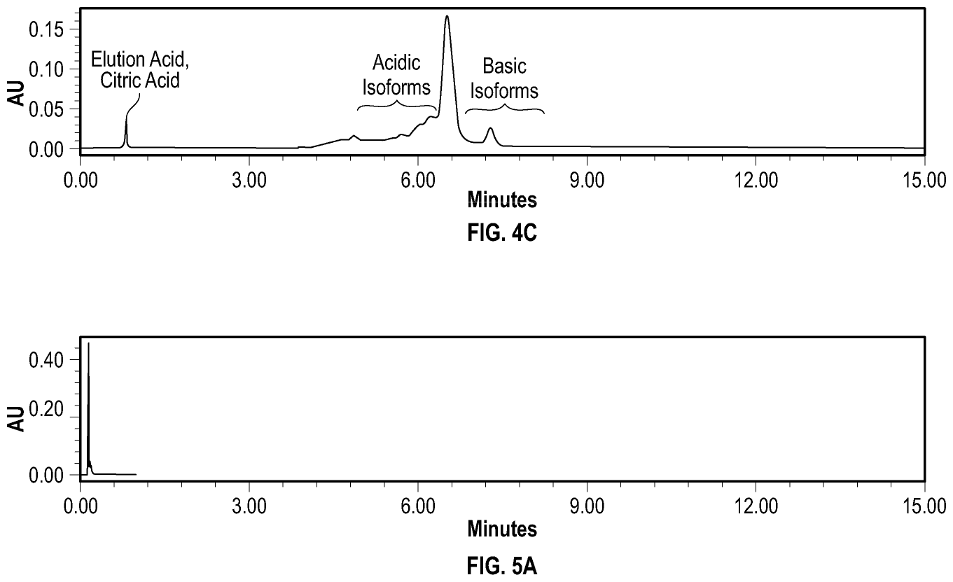

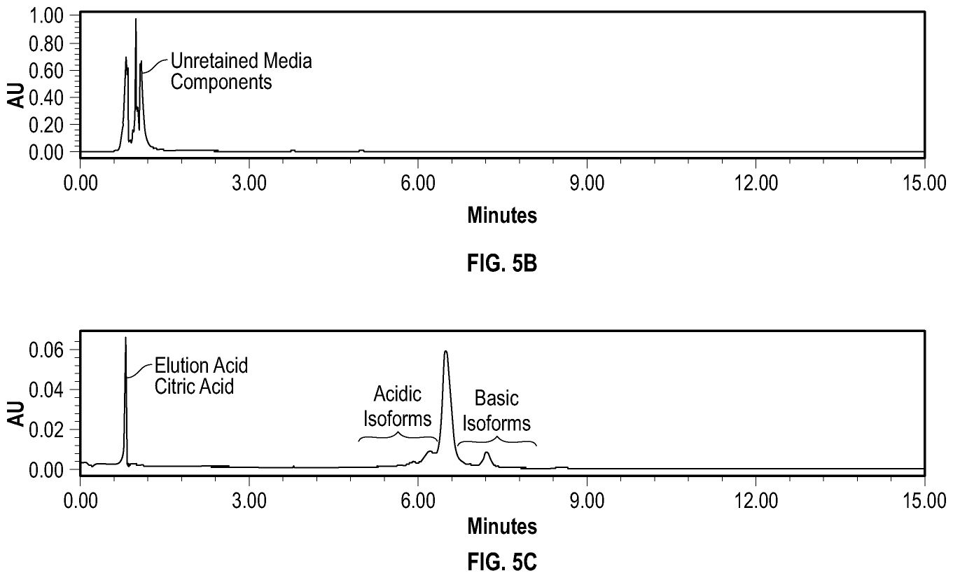

This example describes a method for purifying and characterizing a monoclonal antibody expressed in Chinese Hamster Ovary (CHO) culture media. The process uses a Protein A affinity (ProA) column directly coupled in-line to a strong cation-exchange (SCX) column. CHO culture media may be derived from a CHO cell line engineered to express recombinant monoclonal antibodies. A Protein A affinity column (Premier, 2.1×20 mm, available from Waters Corporation, Milford, MA) is packed with 3.5 μm nonporous divinylbenzene/polystyrene particles bearing immobilized Protein A. This affinity column connects in direct fluidic series to a 4.6×100 mm BioResolve SCX column (available from Waters Corporation, Milford, MA). The column pair is connected to an ACQUITY Premier high-performance liquid chromatography system (Waters Corporation, Milford, MA) equipped with a quaternary solvent manager and a TUV detector. UV detection is carried out at 280 nm. Comparative analyses are performed using affinity-only columns. For the affinity-only control (i.e., results provided in FIG. 4A and FIGS. 5A), 1μL of sample is loaded onto the Protein A column using 20 mM MES, pH 6.8, at a flow rate of 0.6 mL/min for 1 min. Elution is achieved by a second injection of 2 μL of 0.1 M citric acid at a similar flow rate and runtime to those of the first injection. The results of the affinity-only columns are then compared to direct-connect columns at two different points in the method. That is, FIG. 4B and FIG. 5B provide the results from the first injection of sample run with 20 MES pH 6.7, without an IEX gradient; whereas the results provided in FIG. 4C and FIG. 5C illustrate the results after injection of an elution solvent and the use of the IEX gradient. This experiment utilizes an ion-exchange gradient with Mobile Phase A (20 mM MES adjusted to pH 6.8 with sodium hydroxide) and Mobile Phase B (20 mM MES containing 1 M sodium chloride, pH 6.8). Chromatography is performed at 0.6 mL/min with this gradient: 92% A held for 2 minutes, a linear transition from 92% to 89% A over 10 minutes, a 0.5-minute hold at 89% A, then a return from 89% to 99% A over 2.5 minutes, with a total runtime of 15 minutes.

Two sample types are evaluated under these conditions: 2.5 μg/μL NISTmAb spiked into non-transfected CHO media, and a monoclonal antibody produced in CHO media. (Media and sample obtained from Rockland Immunochemicals, Limerick, PA.) For the combined affinity-IEX method, 10 μL of CHO media containing the monoclonal antibody or NISTmAb is injected onto the Protein A affinity column. This column is directly in line with the BioResolve SCX column. Binding and washing are performed with 20 mM MES, pH 6.7, at a flow rate of 0.6 mL/min for 15 minutes, allowing all unbound species to migrate from the affinity column into the SCX column and into waste. Elution from the affinity column is performed by a single injection of 2 μL of 0.1 M citric acid. The eluted analyte is transferred directly to the SCX column, where salt-based separation follows the previously described SCX gradient protocol.

The results provided in FIGS. 4A-4C (NISTmAb spiked into non-transfected CHO media) and in FIGS. 5A-5C (mAbs produced in CHO media) illustrate the advantages of the direct connect method. FIGS. 4A and 5A illustrate the loading and elution from the ProA column, such as the ProA column positioned upstream of the SCX column in a direct connect embodiment of the technology. FIGS. 4B and 5B provide the results after a first injection on a direct connect ProA-SCX column. FIGS. 4B and 5B illustrate that after the first injection, no evidence that any media components were retained on the SCX column. FIGS. 4C and 5C provide results after a second injection in which citric acid is used for elution from the ProA column followed by the salt gradient for IEX chromatography. These results indicate that direct coupling of the affinity (i.e., ProA) column and the IEX (i.e., SCX) column enables quantitative analysis of both titer and charge heterogeneity without additional sample preparation. The advantages provided by this direct coupling (i.e., elimination of a separate sample preparation step) include reduction in analysis time and minimizing sample loss. In the injection-elution method, CHO cell culture media components pass through both columns (i.e., connected affinity and IEX columns) prior to injection of the monoclonal antibody elution, ensuring that the media components do not interfere with IEX separation, providing yet another advantage to the method of the present technology.

1. A method of purifying a target analyte, the method comprising:
a) loading a sample comprising the target analyte onto an affinity chromatography column in direct fluidic connection to an ion exchange chromatography (IEX) column, the affinity chromatography column comprising:
a plurality of nonporous polymer particles having an average particle size between 1.0 μm to 10 μm; and
one or more affinity agents conjugated to a surface of each particle of the plurality of nonporous polymer particles;
and the IEX column comprising:
a plurality of IEX particles having an average particle size of between 1 μm and 10 μm and a charged surface, wherein the plurality of particles are nonporous, porous, or superficially porous and the plurality of IEX particles comprise an inorganic material, an organic material, or a hybrid organic-inorganic material;
b) eluting the target analyte from the affinity chromatography column with an affinity buffer directly onto the IEX column; and
c) eluting the target analyte from the IEX column with an IEX buffer;
wherein the IEX buffer comprises the affinity buffer and one or more additional components.

2. The method of claim 1, wherein the one or more affinity agents are conjugated directly to a surface of each particle of the plurality of nonporous polymer particles.

3. The method of claim 2, wherein the one or more affinity agents are conjugated to the surface of each particle of the plurality of nonporous polymer particles covalently.

4. The method of claim 1, wherein the one or more affinity agents are conjugated indirectly to the surface of each particle of the plurality of nonporous polymer particles through one or more conjugating molecules.

5. The method of claim 4, wherein the one or more conjugating molecules comprise streptavidin.

6. The method of claim 5, wherein the one or more affinity agents are biotinylated.

7. The method of claim 1, wherein the one or more affinity agents are an immunoglobulin-binding protein, an antibody or antigen-binding fragment thereof, or an oligonucleotide.

8. The method of claim 7, wherein the immunoglobulin-binding protein is Protein A, Protein G, Protein A/G, Protein L, or a binding domain thereof.

9. (canceled)

10. (canceled)

11. The method of claim 1, wherein the affinity buffer comprises sodium phosphate, ammonium acetate, or ammonium formate.

12. The method of claim 1, wherein the affinity buffer has a pH of from about 6.8 to about 8.

13. The method of claim 11, wherein the affinity buffer further comprises an organic solvent.

14. (canceled)

15. (canceled)

16. (canceled)

17. The method of claim 1, wherein the IEX buffer comprises a salt.

18. (canceled)

19. (canceled)

20. The method of claim 1, further comprising repeating step (b) one or more times, wherein each repetition of step (b) is followed by one or more repetitions of step (c).

21. (canceled)

22. (canceled)

23. (canceled)

24. The method of claim 1, wherein the direct fluidic connection is a zero dead volume union.

25. A chromatographic system comprising:
(a) an affinity chromatography column and an ion exchange chromatography (IEX) column, wherein the affinity chromatography column and the IEX column are connected via a zero dead volume union, wherein the IEX column is positioned downstream of the affinity chromatography column; wherein the affinity chromatography column comprises:
a plurality of nonporous polymer particles having an average particle size between 1.0 μm to 10 μm; and
one or more affinity agents conjugated to a surface of each particle of the plurality of nonporous polymer particles
and the IEX column comprises:
a plurality of IEX particles having an average particle size of between 1 μm and 10 μm and a charged surface, wherein the plurality of particles are nonporous, porous, or superficially porous and the plurality of particles comprise an inorganic material, an organic material, or a hybrid organic-inorganic material;
(b) a column injector positioned upstream of the affinity chromatography column; and
(c) tubing in fluidic connection with the IEX column positioned downstream of the IEX column.

26. The chromatographic system of claim 25, wherein the one or more affinity agents are conjugated directly to a surface of each particle.

27. The chromatographic system of claim 26, wherein the one or more affinity agents are conjugated to the surface of each particle covalently.

28. The chromatographic system of claim 25, wherein the one or more affinity agents are conjugated indirectly to the surface of each particle through one or more conjugating molecules.

29. The chromatographic system of claim 28, wherein the one or more conjugating molecules comprise streptavidin.

30. The chromatographic system of claim 29, wherein the one or more affinity agents are biotinylated.

31. (canceled)

32. (canceled)

33. (canceled)

34. (canceled)

35. (canceled)
